(19) United States
(12) Patent Application Publication
(10) Pub. No.: US 2006/0020161 A1
(43) Pub. Date: Jan. 26, 2006

(54) Method and device for auditory stimulation for therapeutic application

(76) Inventors: James Mageras, Darien, CT (US); John Klett, Carmel, NY (US)

Correspondence Address: KENYON & KENYON, One Broadway, New York, NY, 10004, US

(21) Appl. No.: 10/896,240

(22) Filed: Jul. 20, 2004

(51) Int. Cl. A61M 21/00

(52) U.S. Cl. 600/28

(57) ABSTRACT

A system to deliver a sound of based therapy having a waveform generator device which is configured to generate signals which are therapeutic to a patient, and a sound delivery system configured to receive signals from the waveform generator device and transmit the signals to the patient, wherein the waveform generator device contains a computational device to internally generate the signals based on mathematical algorithms.

### FIELD OF THE INVENTION

The present invention relates to devices and methods which use sound to stimulate an individual. More specifically, the present invention provides methods and devices for generating waveforms to aid in medical and therapeutic treatment of individuals.

### BACKGROUND INFORMATION

There are several varieties of stimulation devices and techniques that use sound to treat individuals. These devices are targeted to the medical and relaxation fields, including the treatment of tinnitus, commonly known as “ringing of the ears.” The auditory stimulation devices and techniques used in the treatments of tinnitus have serious drawbacks which limit their overall effectiveness.

Often, treatments and therapies which stimulate the auditory senses of patients are conducted in a controlled setting (“in-office”) using complex and expensive equipment and devices. The devices are operated by trained personnel in order to maximize the treatment effectiveness. There are several advantages to providing therapies and treatments in such controlled “in-office” environments. First, the therapies and treatments may be repeated in a more controlled manner, thereby establishing a consistent patient treatment over time. To achieve this consistent treatment, however, patients are required to make appointments and stop at the physician's office for their scheduled treatment. The treatment activity is logged and regulated by the doctor, thereby providing a permanent record of the treatments dispensed. Patients can further be questioned as to their perceptions of the effectiveness of the treatment. Consequently, the physician and/or operator of the equipment can determine whether or not the patient is benefiting from treatments.

A disadvantage of restricting therapies or treatments to an “in-office” setting is that many potential beneficiaries of such treatments or therapies may not be able to schedule time away from work or other activities to take advantage such treatments. As a result, the treatments/therapies are not available for all individuals who may benefit from such treatment. Moreover, the devices used are expensive and cannot be purchased or operated by the average untrained individual, further limiting the availability of these treatments.

The current systems used for control and amelioration of tinnitus, however have significant drawbacks. These tinnitus amelioration control systems treat the affliction through the playback of prerecorded tones on a playback device. The sounds to be provided to the patient are recorded on compact cassettes or on compact disks and are played through a portable delivery system (such as a portable cassette player or portable compact disk player). When played through such portable devices, for example a portable compact disk system, the current tinnitus amelioration protocols produce limited benefit for the individual afflicted with tinnitus because of the inherent limited playback capabilities of the portable devices and the recording medium. The portable devices (i.e. players) do not have sufficient speed accuracy which in turn affects pitch and frequency. The compact disk players also do not provide proper audio fidelity or a capability to minimize distortion and level control accuracy (loudness) to accurately provide the needed tones to patients. The portable devices do not have proper waveform accuracy and harmonic purity. These devices may deliver signals, but when quantitatively measured, the signals differ from the known true values needed for treatment effectiveness. All of these devices also do not provide sufficient capability to regulate the tones and waveform properties generated over a defined time period.

To provide an accurate tinnitus treatment for an individual, the playback device must have sufficient speed accuracy that is very carefully controlled. Portable compact disk units, for example, do not have proper speed control and therefore do not provide an optimal treatment device for patients. A second requirement for accurate treatment of tinnitus is that audio fidelity be maintained while keeping distortion to a minimum. Audio fidelity must be carefully controlled during playback of prescribed waveforms in order to accurately deliver the needed waveforms to the patient. Again, portable compact disk players do not currently possess audio fidelity characteristics that are required for completely accurate patient treatment. Home compact disk playing units also vary excessively in speed accuracy and audio fidelity making their use problematic for patients. Compact cassette devices also have these drawbacks, hampering these units ability to accurately treat patients. Moreover, compact cassette recordings degrade mechanically with the passage of time and usage, therefore limiting the usefulness of the treatment provided. The tape used in the cassettes, for example, often degrades by stretching and other known degradation forms. Compact disk players also possess a significant amount of digital noise which affect the overall signal provided to an individual. The dynamic range of the compact disk players is not sufficient from optimum values for an person seeking stimulation. Compact disk players also do not provide a response which is accurate for the absolute frequency required to be delivered to an individual.

An additional drawback of compact disk based units and compact cassette units is that these systems do not allow control of the treatments dispensed once the patient leaves the treatment providers premises. No data is retained about the number of treatments dispensed, or the times of treatment. Patient feedback is not obtained and as a consequence, patient compliance with treatment protocols is not checked.

There is currently a need to provide a device that will have accurate speed playback and superior audio fidelity to deliver waveforms to individuals desiring to receive these waveforms.

There is also a need to provide a device that will provide for auditory stimulation of individuals to promote therapeutic results for patients.

There is also a need to provide a device that will stimulate the auditory system of individuals while maintaining proper audio fidelity during a treatment program.

There is also a need to provide a portable device which will stimulate an individual, such as through a patient's auditory system as a non-limiting example, while maintaining proper waveform fidelity during the treatment program as well as pitch accuracy to maximize a tinnitus treatment protocol.

There is a further need to provide a device that can be used to treat tinnitus and which provides individuals with ease of use while maintaining high quality levels of treatment and control by a physician or health care provider.

### SUMMARY

It is therefore an objective of the present invention to provide a device that will have accurate speed playback and superior audio fidelity to deliver waveforms to individuals desiring to receive these waveforms. It is also an objective of the present invention to provide a device that will provide for auditory stimulation of individuals to promote therapeutic results for patients.

It is a further objective of the present invention to provide a device which will stimulate the auditory system of individuals while maintaining proper audio fidelity during a treatment program.

It is a still further objective of the present invention to provide a portable device which will stimulate an individual, such as through a patient's auditory system as a non-limiting example, while maintaining proper waveform fidelity during the treatment program as well as pitch accuracy to maximize the tinnitus treatment protocol.

Moreover, it is an objective of the present invention to provide a device that can be used to treat tinnitus and which provides individuals with ease of use while maintaining high quality levels of treatment and control by a physician or health care provider.

The objectives of the present invention are achieved as illustrated and described. The invention provides a system to deliver a sound based therapy. The invention comprises a waveform generation device which is configured to generate signals which are therapeutic to a patient, and a sound delivery system configured to receive signals from the waveform generator device and transmit the signals to the patient, wherein the waveform generation device contains a computational device to internally generate the signals based on mathematical description or algorithms, wherein the algorithms may be introduced via software.

The invention also provides a method of providing a waveform stimulus to an individual. The method recites providing a waveform generator, and generating a waveform stimulus using the waveform generator, wherein the generating of the waveform stimulus is performed according to algorithms provided to the waveform generator, and delivering the waveform stimulus to the individual.

The invention also provides a method of providing an waveform stimulus to an individual. This method provides for loading a treatment protocol with a number of treatments into a memory of a waveform stimulus system, wherein the treatment protocol includes at least one algorithm to be used by a waveform generator to produce a waveform and a number of treatments to be dispensed to an individual, initiating a treatment from the waveform generator to stimulate the individual, wherein the treatment is based upon the at least one algorithm, and decrementing the number of treatments which may dispensed by the waveform generator.

The invention may be used to deliver sound and sound based therapies or treatments in a precisely defined signal or waveform in both static and dynamic formats. The invention can be used for psychiatric applications, self-hypnosis and meditation. The invention can also be used to stimulate neuron growth in individuals.

### BRIEF DESCRIPTION OF THE DRAWINGS

FIG. 1 is a plan view of a system for stimulating the auditory system of individuals for therapeutic treatment.

### DETAILED DESCRIPTION

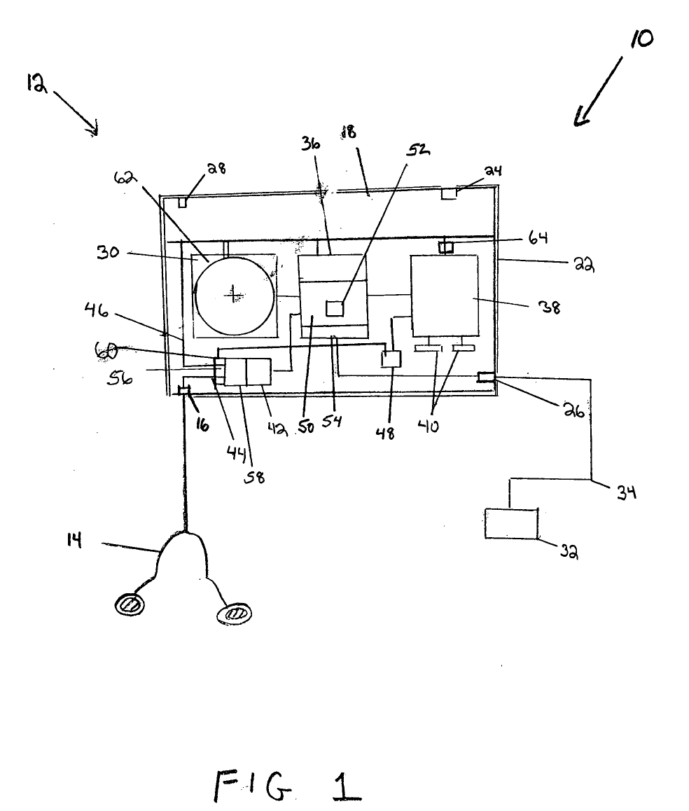

Referring to FIG. 1, a system 10 for stimulating the auditory senses of individuals is illustrated. The system 10 may be prescribed by a physician for treatment of auditory ailments. The system 10 delivers audible signals, as a non-limiting example, to patients who may benefit from delivery of the waveforms to the ears to treat, for example, tinnitus. The system 10 is capable of delivering treatments and therapies that require precise control of frequency, harmonic content, waveform purity and/or loudness. The system 10 may be comprised of individual components so that components may be switched and/or tailored according to the needs of the patient and/or the treatment provider. The system 10 is primarily designed to be portable so that patients may receive treatment at any location desired, rather than at a health care provider facility for example. The system 10 can also be designed to interface with a stationary system (as a centralized server based system) for uploading and downloading information to and from mobile units. The system 10 is comprised of a portable audio waveform generation device 12 and a sound delivery system 14. The portable audio waveform generation device 12 and an associated input/display screen 38 are activated through an on/off button 64 placed on a casing 22 of the system 10. The portable audio waveform generation device 12 is composed of components placed inside the casing 22 for easy transport of the waveform generation device 12. The casing 22 may be a sealed unit so that tampering of the device 12 by unauthorized persons is prevented. The casing 22 may be made of a high density polymer plastic or similar rugged material, for example, that will prevent conduction of electricity external to the device 12 as well as adding impact resistance in the event the waveform generation device 12 is dropped or struck by an object. The casing 22 may provide a shielding capacity to limit internal and external influence of electrical components on the system 10. The casing 22 may be constructed to be weatherproof, thereby providing resistance to moisture intrusion.

The device 12 has an internal battery 18 to power the internal electrical components housed in the casing 22 as well as components attached to the system 10. The battery 18 is sized such that a proper number of treatments is dispensed by the system 10 according to the needs of the patient. The battery 18 may be configured in any arrangement, however, in the illustrated embodiment, the battery 18 is a rechargeable unit. The battery 18 may be recharged through connection of a recharger connected through a recharger connection 24, or through a USB or firewire system. The recharger connection 24 may be a standard connection or may be a specialized connection, requiring the patient to return to the health care provider to recharge the system 10 after discharge. Battery types may include, but not be limited to, nickel-cadmium or lithium-ion rechargeable units. The amount of charge of the battery 18 may be displayed on an input/display screen 38 or through a battery charge indicator 28 located on a side of the casing 22. The recharging capacity of the battery 18 may be either a trickle charger or a fast rate charger. The battery 18 may also be sized such that only one treatment dispensation may be provided before recharging is again required for the system to limit a person from dispensing two treatments “back to back”, thus overtreating him or herself. The battery 18 may also be designed to be only recharged after a predetermined recharge time, for example one day, so that a patient may not obtain treatment more than once over a predetermined interval of time.

In the event of a battery malfunction or if the battery 18 is drained completely, power to the memory components of the system 10 may be maintained through an internal back-up battery 20, thereby preventing data loss. The internal back-up battery 20, as illustrated, is a button design unit, however any configuration may be used. The internal back-up battery 20 may be a rechargeable unit, however as illustrated, the back-up battery 20 is a single use/disposable unit. The back-up battery 20 may be stored in a separate compartment 62 in the casing 22. The separate compartment 62 may be accessed through a door cover 30. The door cover 30 may be a hinged or sliding unit.

Digital signals are produced by a waveform generator 42. Although possible to store a digital signal for playback, the current invention illustrated in FIG. 1 includes a waveform generator 42 which generates signals using mathematical definitions or algorithms which are specified according to the needs of the patient. The waveform generator 42 may produce digital signals over a wide sound spectrum, such as infrasound to ultrasound waveforms, and consequently is capable of treating patients with varying treatment needs. The waveform generator 42 may be a microprocessor unit (MPU) or a digital signal processor (DSP) as non-limiting examples. The waveform generator 42 may also be in the form of an analog circuit which is digitally controlled by a computational device such as a microcontroller unit (MCU). The applicable, non-limiting units which may be used in conjunction with the waveform generator 42 or as the waveform generator include an Analog Devices model ADSP-21060/ADSP 21060L DSP, a Motorola model DSP56362, DSP56364, DSP56366, or DSP 56367 or an Analog Devices Blackfin model BF535, BF533 or BF561.

The signals produced by the wave form generator 42 may be sent to a digital/analog converter 58. The signals exiting either the digital/analog converter 58 or the wave form generator 42 may enter a low distortion signal amplifier to provide sufficient signal amplification. The digital to analog converter may be a high-resolution type unit, for example, a device from Burr Brown model DAC 1220-20-bit low power digital to analog converter, an Analog Devices model AD 1871, stereo audio, 24 bit, 96 kHz, Multi-Bit Sigma Delta ADC, or a Cirrus Logic CS 43122, high performance 24-bit, 192 kHz stereo digital-to-analog (D/A) converter. The digital to analog converter 58 may be connected to the battery 18 through a connector 44 with attached line 46. The system 10 may also provide a digitally controlled level control to scale the output level from the digital to analog converter for maximum resolution at lower volume levels. Non-limiting examples of such controllers are a Maxim-Max5419 100k ohm, 256-Tap, nonvolatile I2C Interface Digital Potentiometer, an Analog Devices AD5160—256 Position SPI compatible digital potentiometer and a Xicor Z9455—dual two-wiper digitally-controlled potentiometer. The system 10 may also include a low distortion analog signal output amplifier that will accurately drive transducers, such as earphones or headphones directly. The low distortion signal can be further amplified to drive speakers or other transducers as desired by either the treatment provider or the patient. Additionally, self-powered transducers may be used such as battery powered speakers. Non-limiting examples of low distortion analog signal output amplifiers include a National Semiconductor LM 4808 Dual 105 mW headphone amplifier, a Fairchild FAN7005 200 mW stereo headphone amplifier or a National Semiconductor LM4864 300 mW audio power amplifier. The amplified signals are then emitted out of the casing 22 to head phones or earphones 14 which are connected to the casing 22 through a jack 16.

Users of the system 10 may be provided with an input/display screen 38 which is configured to display information to the user about the current status of the system 10. The input/display screen 38 may take input from an input apparatus, in the current example buttons 40. The input/display screen 38 may also be configured as a touch screen or may accept handwriting through a handwriting recognition system. The input buttons 40 are configured to allow a user to respond to inquiries from the system 10 regarding needed user input. Input to the system 10 may also be through speech recognition, wherein a microphone preamplifier, analog to digital converter and speech recognition software can be included. Information which may be displayed on the screen 38 to the user may be, for instance, the number of treatments provided, the number of treatments to be dispensed, the battery charge level, the amount of time left in an ongoing treatment, healthcare provider information and volume level. The input/display screen 38 may take information from a processor 50 which may also be used as the waveform generator 42 or may be a separate processor. The processor 50 provides available information for display to the user allowing the user to initiate menu selections and treatment. The input/display screen 38 may be a liquid crystal display to minimize power drain on the battery 18. The input/display screen 38 and the input buttons 40 may be illuminated to provide a user with the capability to operate the system 10 in low light levels. Information can be obtained from a user through the use of the input/display screen 38. Inquiries such as a series of questions recording the impressions of the user after treatment can be obtained through screen prompts, where the user inputs information that is stored in non-volatile memory. Other configurations may be used besides input buttons 40 for input of information such as, but not limited to, electromechanical switches, membrane switches, capacitive sensing touch switches and mini-keypad switches. Touch pads or buttons on the device may incorporate Braille or provide a tactile interface. The input/display screen 38 may be a liquid crystal display, for example, or a light emitting diode in single or matrixed configuration.

A speaker 48 may be connected to the input/display screen 38 to provide auditory feedback to the user as to when an input has been made (i.e. the speaker may “chirp” when an input is made or may prompt the user when a treatment is to be dispensed). The speaker 48 may be configured to provide minimal drain on the battery 18. The battery 18 may also be disconnected from the speaker 48 and the rest of the unit at the desire of the user, thereby eliminating further power drain. The speaker 48 may also be connected to a low distortion signal amplifier 50 to provide required gain of sound levels to the speaker 48. As a consequence, waveforms generated by the waveform generator 42 may be played through the speaker 48 and used for treatment of the afflicted individual. The speaker 48 may also be configured to provide an auditory signal to the user when a computer 32 or other device is connected to the casing 22, thus providing the individual with the ability to ascertain the system status (i.e. if the system 10 is connected to a centralized server or is downloading information to a connected computer). The speaker 48 can be used in conjunction with a speech synthesizer to generate treatment signals or allow the unit to be operated by the visually impaired. When used in the treatment of tinnitus, for example, headphones/earphones may be used to deliver the desired waveforms. When used for meditative, relaxation and psychiatric applications, the internal or external speakers may be used to deliver the waveform. The transducers used, therefore, can be matched to the desired output for maximum patient treatment response.

A processor 50 is located in the casing 22 to perform needed calculations and provide and receive information to and from the waveform generator 42, the non-volatile memory 36 and the input/display screen 38. The memory 36 may be a serial and parallel flash memory, for example a Samsung KM29W8000-NAND Flash Memory, or and Intel 28F640W30, 28F320W30 or 28F128W30 Flash memory. Battery Supported SRAM such as a MAXIM/DALLAS DS 1230W—NV SRAM with self-contained lithium energy source and control circuitry, a STMicroelectronics-M48Z129—low power SRAM with self-contained battery or a STMicroelectronics—M48Z32V—low profile 44-pin SOIC with battery external to the package may also be used. Additionally, a phase-change memory (PCM) may also be used.

The processor 50 may receive information stored in the non-volatile memory 36, such as the number of treatments left to be dispensed and prompt the user through the input/display screen if the user wishes to commence a treatment. The processor 50 may then accept user input through, for example, input buttons 40, to begin a treatment. The processor 50 may then execute a command to activate the waveform generator 42 to produce signals. Completion of the treatment may be determined through a real time clock 52 or through completion of the mathematical algorithm. The processor 50 may then write information into the non-volatile memory 36 regarding the dispensation of the treatment, as well as the time and day the treatment was dispensed. Information may be stored or downloaded in the non-volatile memory 36 by the actuation of the processor 50. In either case, information from the processor 50 or entering the processor 50 may pass through an encryption unit 54. The purpose of the encryption unit 54 is to send and receive encrypted data to an apparatus connected to the system 10 through communications port 26. The communications port 26 may be a standard universal serial bus (USB) connection or other standard connection for transferring data to and from computing units. The communications port 26 may be a USB interface port and chipset, such as from National Semiconductor USBN9603/4 integrated USB node controller with USB transceiver, 3.3 V re, serial interface engine (SIE), USB endpoint, FIFO's 8-bit parallel interface and clock or a Cypress Semiconductor SL811HS USB host/slave controller USB serial Interface with internal full/low-speed transceivers. A fire wire interface port and chipset may also be used, such as a Texas Instruments TSB43AA82A (iSphynxII): 1394 Integrated PHY and Link Layer Controller or a NEC pPD72873 IEEE 1394 combo (link+physical layer) controller.

An internal clock, different than clock 52, may be used to provide a highly accurate timebase used for the generation of waveforms and signals. The internal clock may be temperature compensated crystal oscillator (TCXO) with an accuracy better than 1 part per million, a GPS receiver that receives time base information from the global positioning satellite system or a rubidium plasma atomic clock with an accuracy better than 20 parts per trillion with jitter specs in the less than 1 picosecond range. Jitter may be removed by use of an analog circuit or a synthetic phase lock loop. The use of a global positioning system clock signal may be used as both the clock 52 signal as well as the internal clock to regulate treatment parameters.

A computer 32 may be connected to the system 10 through a cable 34 to upload and download information from the non-volatile memory 36. The computer 32 may also transfer data to the system 10 such as the number of treatments to be dispensed, as well as the mathematical algorithms to be used to impart sounds to the patient. The computer 32 may also update system information, such as the clock 52 as necessary. Volume control for the system 10 may be performed through a volume control arrangement 56 connected to the low distortion signal amplifier 60. The volume control arrangement 56 can be a preset arrangement if desired by the provider to accurately control the dispensed sounds, or the volume control arrangement 56 may be enabled such that the user may have control over the dispensed sounds or the volume control may be accomplished through the computer 32. The computer 32 may operate through the use of software that presents an easy to use graphical user interface, thereby allowing a trained operator to control, program, exchange data with and regulate the use of a portable audio or sound based treatment device 10. Software on the computer 32 may also be used to aid in patient characterization by controlling the system 10. The computer 32, in turn, may send and receive information to and from a centralized server to allow data, such as user information and software updates to be shared between the server, the computer and the system 10.

The system 10 may also be configured to interact with the centralized server (i.e. a unit which is stationary or based within a physician's office). Connection of the system 10 to other computers may be through the Internet, dedicated phone line, cable connection or other similar communication pathway. The centralized server may be equipped with a graphical user interface to allow simplified dispensation of treatment. If the system 10 is configured to interact with a centralized server, the centralized server may use internet protocol networking or telephone/modem (dialup) technologies to communicate via a clear or encrypted line. Software updates for the practitioners in-office treatment control computer and the treatment devices distributed may be distributed through dialup technologies, for example. The centralized server may control distribution of proprietary software, deliver the software to individual computers or systems 10 and download information from computers or systems 10 which have accumulated data on patient treatments. The system 10 may also be configured to interface with the computer 32 or the centralized server through accessing an internet web page through cable, phone or WiFi connection.

Other configurations of components may also be used to accomplish the provision of a waveform. A communication port may be connected to a digital signal processor which in turn sends and receives information to and from a connected memory. The output of the digital signal processor may be connected to a digital to analog converter which in turn is connected to a level shift control. The level shift control may then be connected to an amplifier to amplify the waveform. The digital signal processor may also be connected to the level shift control. Input/output channels may be provided to this configuration, for example to the digital signal processor, for inputting and outputting information.

A communication port may also be connected to a microprocessor unit that in turn is connected to both a memory and a digital signal processor. The output of the digital signal processor may be connected to a digital to analog converter and attached attenuator (level shift) and an amplifier to output the desired waveform. The microprocessor unit may also be connected to the attenuator.

A communication port may also be connected to a central processor unit which is in turn connected to a memory. The digital signal provided by the central processor unit may then be received by a digital to analog converter and attached attenuator and amplifier for production of the waveform.

A communication port may also be connected to a microcontroller unit which is also connected to a memory. The microcontroller unit may also be connected to analog generator circuits by a digital to analog converter. The output of the analog circuit may then connected to an attenuator, as a non-limiting example.

Operationally, a user obtains a system 10 from a physician, for example. The physician diagnoses the auditory ailment of the patient and prescribes a treatment protocol for the individual. The system 10 is programmed through the use of a computer to generate waveforms to the individual based upon mathematical description or algorithm downloaded into the system 10. The user places headphones (or earplugs/speakers or other transducers) on the ears (as a non-limiting example) and initiates characterization (i.e. choosing the right waveform) and then treatment through actuation of the system 10. Characterization may also be accomplished in a physician's office, separate from the system 10. A counter (which allows a prescribed number of treatments) is then decreased in number after completion of each treatment. After treatment, the user is asked a series of questions regarding observations from the treatment. The system 10 retains the time and date of treatment and the observations from the treatment for eventual download at a physicians office. After a predetermined time, the system 10 allows the user to dispense another treatment. After the treatment protocol is completed, the user is then reminded by the system 10 that all treatments are completed or will be soon completely dispensed and that the patient should return to the physician's office for evaluation. The patient then returns to the physician's office with the system 10 and the information retained in the memory of the system 10 is then downloaded for record retention by connecting the system 10 to a computer 32. The physician may then prescribe additional treatments if necessary by adjusting the counter in the system 10. The system 10 may also perform an ongoing adjustment of the treatment protocol based on patient data entry. The centralized server may gather information from other systems for logging and tracking of groups of individuals and updating of software.

The current apparatus and method allow a provider of services, such as a health care provider, to provide an accurate treatment for an individual with tinnitus, for example. The device used in the current invention has sufficient frequency accuracy (pitch) that is very carefully controlled as the components in the system 10 are designed to maintain accurate pitch, unlike the drawbacks of other systems. The pitch control of the system 10 allows the health care provider to accurately control the pitch delivered to the patient, maximizing the effectiveness of the treatment.

The current apparatus and method provide a significant benefit over portable compact disk units, for example, because the portable compact disk units have excessive variation in pitch and therefore do not provide an optimal treatment base. The current apparatus also provides superior audio fidelity (distortion) control, as compared to compact disk units and compact cassette devices. The current apparatus provides a system that provides a pure waveform while minimizing noise. The dynamic range of the current apparatus is maintained while the noise is minimized, therefore limiting unneeded wave input into an individual. The current apparatus and method also provide superior speed accuracy and audio fidelity compared to compact disk and compact cassette devices. The current apparatus and methods are simplified such that treatments are easily dispensed to afflicted individuals.

The compact size and light weight of the portable devices allows the device to easily transported and stored easing demands on the user.

The apparatus is designed to interact with a centralized server according to the current invention to offer superior audio generation capabilities, while providing the additional economic benefit of minimizing overall costs per treatment and allowing for data gathering from multiple sources (multiple systems) to compile treatment records over a population of individuals.

The current apparatus and method may also be used to provide waveforms to individuals for relaxation therapy, to improve a patients mental focus, provide a treatment for attention deficit disorder, reduce epileptic episodes of individual, induce sleep, block pain or change the mental or physical state of an individual, wherein the individual would benefit from a waveform produced by the system 10. The current apparatus and method may also be used to train musicians to properly recognize a “correct” waveform or tone by producing a high quality waveform for reference. The system 10 may also provide a waveform to an individual on other areas of the body besides the ears to stimulate the area receiving the energy, such as muscles and bones of individuals, wherein the transducers may be electromechanical devices to stimulate (for example vibrate) such body parts. Moreover, the current apparatus and method may be used to treat other conditions which affect a patient's hearing, including, but not limited to, mental disorders.

In the foregoing specification, the invention has been described with reference to specific exemplary embodiments thereof. It will, however, be evident that various modifications and changes may be made thereunto without departing from the broader spirit and scope of the invention as set forth in the appended claims. The specification and drawings are accordingly to be regarded in an illustrative rather than in a restrictive sense.

What is claimed is:

1. A system to deliver a sound based therapy comprising:
a waveform generator device which is configured to generate signals which are therapeutic to a patient; and
a sound delivery system configured to receive signals from the waveform generator device and transmit the signals to the patient, wherein the waveform generator device contains a computational device to internally generate the signals based on mathematical algorithms.

2. The system according to claim 1, wherein the computational device is a microcontroller unit.

3. The system according to claim 1, wherein the computational device is a digital signal processor.

4. The system according to claim 1, wherein the computational device is a microprocessor unit.

5. The system according to claim 1, further comprising:
a non-volatile memory, wherein the computational device is connected to the non-volatile memory such that data may be exchanged between the computational device and the memory.

6. The system according to claim 1, further comprising:
a computer with a user interface wherein the computer is configured to interface with the waveform generator device.

7. The system according to claim 1, wherein the sound delivery system is one of earphones, headphones and speakers.

8. The system according to claim 6, wherein the computer is configured to exchange data and control operation with the waveform generator device.

9. The system according to claim 6, further comprising a centralized server, wherein the computer is configured to interface with the central server, the centralized server configured to upload and download information from the computer and a non-volatile memory connected to the waveform generator device.

10. The system according to claim 1, wherein the waveform generator device includes a real time clock to regulate the treatment to the patient.

11. The system according to claim 1, wherein the waveform generator device is configured to remind a patient to obtain a therapy from the system.

12. The system according to claim 1, wherein the waveform generator device is configured to limit treatment dispensation to prescribed times.

13. The system according to claim 1, wherein the waveform generator device is configured with a counter to dispense a preset number of treatments.

14. The system according to claim 13, wherein the system is configured to accept further treatments for dispensation from a computer.

15. The system according to claim 14, wherein the system is configured with an interface to accept and store information provided by the user.

16. The system according to claim 15, wherein the interface includes a speech synthesizer.

17. The system according to claim 1, wherein the one of the portable audio and sound based treatment signal or waveform generator device has a time base which controls a pitch of sound generated by the system.

18. The system according to claim 1, further comprising a communication port that provides bidirectional data and software exchange between the system and a computer.

19. The system according to claims 15, wherein the interface is configured with at least one of buttons and touch pads.

20. The system according to claim 19, wherein the interface is illuminated to aid in data entry.

21. The system according to claim 1, wherein the system includes a high resolution digital to analog converter to convert data output from the computational device into one of an analog audio signal and a waveform.

22. The system according to claim 21, further comprising a low distortion analog signal output amplifier to drive the sound delivery system, wherein the sound delivery system includes at least one of headphones, earphones and speakers.

23. A method of providing a waveform stimulus to an individual, comprising:
providing a waveform generator;
generating a waveform stimulus using the waveform generator, wherein the generating of the waveform stimulus is performed according to one of an algorithm or algorithms provided to the waveform generator; and
delivering the waveform stimulus to the individual.

24. A method of providing a waveform stimulus to an individual, comprising:
loading a treatment protocol with a number of treatments into a memory of a waveform stimulus system, wherein the treatment protocol includes at least one algorithm to be used by a waveform generator to produce a waveform and a number of treatments to be dispensed to an individual;
initiating a treatment from the waveform generator to stimulate the individual, wherein the treatment is based upon the at least one algorithm; and
decrementing the number of treatments which may dispensed by the waveform generator.

25. The system according to claim 1, wherein the system is portable.
